(19) United States
(12) Patent Application Publication
(10) Pub. No.: US 2008/0097384 A1
(43) Pub. Date: Apr. 24, 2008

(54) Gene Therapy

(76) Inventor: Andrew Pacey, Herts (GB)

Correspondence Address: SCHWEGMAN, LUNDBERG & WOESSNER, P.A., P.O. BOX 2938, MINNEAPOLIS, MN, 55402, US

(21) Appl. No.: 10/594,099

(22) PCT Filed: Mar. 29, 2005
(86) PCT No.: PCT/GB05/01243

§ 371 Date: Sep. 28, 2007

(30) Foreign Application Priority Data
Mar. 25, 2004 (GB) 0406728.6

(51) Int. Cl. A61M 5/00

(52) U.S. Cl. 604/509; 604/522

(57) ABSTRACT

The present invention provides a method for introducing nucleic acid (30) into cells of a region of the human or animal body, which method comprises substantially occluding an efferent vessel from said body region and introducing said nucleic into that body region under pressure via said efferent vessel and further apparatus for introducing nucleic acid into cells of a region of the body comprising: a reservoir for holding a liquid formulation which comprises said nucleic acid; a catheter tube in fluid communication with said reservoir for conveying said liquid formulation to said body region via an efferent vessel of said body region; pressure development means for pressurising the liquid conveyed by the catheter; and occlusion means (8) for substantially occluding said efferent vessel.

The present invention relates to apparatus and methods for the introduction of nucleic acid into a target organ of the human or non-human animal body, in particular into the liver.

While gene therapy is of tremendous potential benefit in the treatment of hereditary and acquired diseases, one of the main hurdles to current gene therapy techniques is the low level of transfection which is seen in the clinics. Gene therapy relies on the animal cells taking up the vector which incorporates the therapeutic nucleic acid as transfection is necessarily a prerequisite to efficient gene expression. Even if the administered nucleic acid is a regulatory rather than a coding sequence it must still be taken up by the cell in order to exert its influence on the cell's protein production.

The central role which the liver plays in the body in terms of protein production and the prevalence of liver cancers makes this organ a key target for gene therapy. However, systemic injection, for example into the vein of the arm (vena mediana cubiti), has not resulted in significant transfection of the liver hepatocytes (Habib et al., Human Gene Therapy 12: 219-226 [2001]). The hepatocytes are the major cell type of the liver and they are responsible for the synthesis, degeneration and storage of a wide range of substances including the synthesis of all plasma proteins except for antibody and transfection of these cells must be achieved if the therapy proposed relates to any normal liver function.

Attempts have been made to make local injection into the hepatic artery (Habib et al. supra and Reid et al., Cancer Research 62: 6070-6079 [2002]) but again the transfection rate of the hepatocytes was highly unsatisfactory.

It has often been proposed to inject tumours directly when the proposed therapy is cancer therapy; however most cancers that recur after surgery or radiation are multifocal and therefore intratumoral injection is not feasible in these circumstances.

Therefore a need exists for new ways of transfecting liver cells at efficiencies which can result in effective gene therapy.

In mice hydrodynamic injection has been used to transfect liver cells (Liu et al., Gene Therapy 6: 1258-1266 [1999]). In this case a large volume of fluid containing the plasmid vector encoding the gene of interest was injected forcefully and fast into the tail vein of the animal. The volume used is 1-2 ml which is equivalent to the total circulation in mice. The formulation of plasmid in saline goes up the vena cava up to the heart. The mouse heart does not cope with this volume and that forces the liquid carrying the plasmid to enter the hepatic veins of the liver. According to this technique, the pressure applied resulted in successful uptake of the plasmid by the hepatocytes due to the fluidity of the cell membrane.

However, such a technique would not be applicable to larger animals such as man where the forceful injection of large volumes of fluid into the systemic circulation would lead to heart failure.

The present invention addresses these problems and utilises hydrodynamic principles to achieve transfection of cells at levels adequate for gene therapy without damaging the heart.

Thus, in one aspect, the present invention provides a method for introducing nucleic acid into cells of a region of the human or animal body, which method comprises substantially occluding an efferent vessel from said body region and introducing said nucleic into that body region under pressure via said efferent vessel.

The region of the body will preferably be an organ but may be any part which can be effectively isolated, in whole or in part, from the normal blood circulation by occlusion of an exiting vein, i.e. occlusion of the region's efferent vessel. Examples of the efferent vessels for various organs and other parts of the body (e.g. limbs) are as follows: the renal vein for the kidney, the adrenal vein for the adrenal glands, the pulmonary vein for the lungs, the coronary vein or sinus for the heart, the splenic vein for the spleen, the femoral vein for the lower limb, the pancreatic vein for the pancreas.

It will be understood that where mention is made of ‘the body’ or ‘a patient’ this includes human and non-human animals, including livestock and companion animals as well as animals used in research; humans are nevertheless preferred subjects.

More particularly the present invention provides a method for introducing nucleic acid into cells of a body organ which method comprises substantially occluding an efferent vessel of said organ and introducing said nucleic acid into the organ under pressure via said efferent vessel. In a specific embodiment the invention provides a method for introducing nucleic acid into liver cells, which method comprises substantially occluding a hepatic vein and introducing said nucleic acid into the liver under pressure via said hepatic vein.

In a further aspect the invention provides apparatus for introducing nucleic acid into cells of a region of the body comprising: a reservoir for holding a liquid formulation which comprises said nucleic acid; a catheter tube in fluid communication with said reservoir for conveying said liquid formulation to said body region via an efferent vessel of said body region; pressure development means for pressurising the liquid conveyed by the catheter; and occlusion means for substantially occluding said efferent vessel.

In a further aspect the invention provides apparatus for introducing nucleic acid into liver cells comprising: a reservoir for holding a liquid formulation which comprises said nucleic acid; a catheter tube in fluid communication with said reservoir for conveying said liquid formulation to the liver of a patient via a hepatic vein; pressure development means for pressurising the liquid conveyed by the catheter; and occlusion means for substantially occluding said hepatic vein.

In a further aspect the invention provides apparatus for introducing nucleic acid into cells of a body organ comprising: a reservoir for holding a liquid formulation which comprises said nucleic acid; a catheter tube in fluid communication with said reservoir for conveying said liquid formulation to the organ of a patient via an efferent vessel of said organ; pressure development means for pressurising the liquid conveyed by the catheter; and occlusion means for substantially occluding said efferent vessel.

This technique can be used to introduce genetic material into any organ of the body other than the brain. The liver is especially preferred and is used in the following discussion to exemplify the technique. However, other suitable organs include the kidney, heart, spleen, pancreas, lung, adrenal glands, stomach, prostate gland, ovary etc. The organ or region of the body requires a blood circulatory system in which occlusion of an efferent vessel (efferent vein), the organ or region is temporarily totally or partially isolated from the normal blood circulation. It will be understood that where mention is made of the liver, hepatic vein etc. that the same techniques and principles apply mutatis mutandis to other organs and regions of the body.

When limbs are treated in accordance with the methods of the invention the cells to be transfected may be of the blood vessels therein, e.g. to increase blood flow in ischaemia which may be achieved by transfection with a plasmid expressing VEGF. Alternatively muscle cells may be transfected with a plasmid encoding a growth factor or as a treatment for muscular dystrophy. The heart is a special case and where it is desired to transfect heart cells, e.g. cardiac myocytes, the heart itself is not occluded but the coronary vein or sinus can be occluded in order to perform gene delivery and transfection.

According to the normal circulatory system blood enters the liver from the hepatic artery and hepatic portal vein and is then collected in one of three hepatic veins (right, central and left) and travels from there to the heart. Thus by substantially occluding one of the hepatic veins the liver may be temporarily and partially isolated from the normal circulation. Importantly, the effect of the occlusion means is that when the liquid formulation comprising the nucleic acid with which it is desired to transfect the liver cells is introduced into the liver under high pressure, the heart is substantially isolated from this liquid. This means the heart cannot be damaged by exposure to high-pressure liquids and means the pressure at the site of delivery in the liver is such that uptake by the liver cells of the nucleic acid is sufficient to allow successful gene therapy. Occlusion of and delivery through the left hepatic vein is preferred according to the present invention. Occlusion of the efferent vessel of other organs and regions of the body has a similar isolating and protective, viz a viz the heart, effect.

The occlusion means could take any suitable known form. It is for example envisaged that a mechanical expansion mechanism, e.g. umbrella style could be employed. However preferably the occlusion means comprises a balloon arranged to expand to conform to the vein wall, e.g. upon being filled with fluid, preferably saline. Balloons are typically made from four basic material families, silicones, polyurethane (PU), polyamide (PA) and latex.

PU can be used to make a very compliant balloon. These can be inflated under low pressure, and a high inflation ratio (can expand over ×300 diameter). The balloon will tend to form to the vessel, rather than forcing the vessel to follow the form of the balloon. PA can be used to make a higher pressure less compliant balloon. These will tend to inflate to form a hard balloon. This offers a solid location or delivery. Silicone and latex can also be used. These offer high inflation ratios and could be fabricated using dip coating. Latex and silicone tend to be less inert than PU and PA. The occlusion means could be provided separately of the catheter tube but is preferably provided integrally thereon.

In some embodiments two or more occlusion means could be provided. This would allow, for example, substantial isolation of the hepatic vein both upstream and downstream of the point of introduction of the nucleic acid. Alternatively, two occlusion means could be used such that the first (closer to the reservoir) means acts as a pressure dam and the second means effects the occlusion. Thus, a more compliant first occlusion means, e.g. a more compliant balloon, takes some of the pressure wave that could be induced during injection; a second balloon acts only as an occlusion device and leakage is minimised. Such a system may be especially desirable where in excess of 200 or 300 ml of liquid is being injected.

The pressure development means could take any convenient form but is preferably operatively associated with the reservoir in order to pressurise the liquid formulation to a predetermined pressure. In a simple convenient example the reservoir comprises an ordinary syringe and the pressure development means an ordinary syringe driver. The syringe driver may then be programmed to deliver the liquid formulation at a predetermined rate which will determine the pressure at which the formulation is administered to the liver for a given catheter lumen bore, aperture size etc. Of course more complicated arrangements are also envisaged which could include for example a pressure sensor to form a feedback loop. The reservoir may comprise a flexible bag, as used in a saline drip for example, which may be provided with a jacket by way of pressure development means which can expel the liquid formulation in a controlled manner. Expulsion can be performed manually.

The reservoir is preferably in the form of one or more syringes. A single syringe can deliver large volumes, e.g. of 300 ml but it may be more convenient to deliver the liquid at the desired pressure to use 2 or 3 syringes, e.g. delivering 150 ml or 100 ml each. Separate syringes allow the convenient co-administration of nucleic acid and a further substance, e.g. a therapeutically active agent. A plurality of reservoir compartments is thus preferred. Typically the liquid from these compartments will be mixed such that the liquid delivered down the catheter is a mixture of the liquid from all compartments. Thus the reservoir compartments are preferably emptied simultaneously but may be emptied consecutively. Having syringes attached to a manifold allows controllable delivery of different fluid types.

Preferably the pressure development means is adapted to allow delivery of the formulation comprising the nucleic acid to the liver under a pressure which is sufficient to cause uptake by the liver cells of the nucleic acid. Suitable pressures include 10-80 mmHg for example 15-50 mmHg, preferred pressures include 20-60 or 30-50 mmHg.

The catheter may be arranged to introduce the nucleic acid into the vein substantially axially, substantially radially, at an intermediate angle or any combination thereof. Radial introduction is presently preferred since this allows occlusion means to be provided on the catheter both up and downstream of the point of introduction, thereby allowing the introduction site to be substantially fully isolated and unaffected by normal blood flow. The preferred location of these injection ports will also depend on the location of the cells which it is desired to transfect. The appropriate size of catheter will depend on the target organ or body region and the vein to be occluded but may conveniently have a circumference of 5-10 mm e.g. 7 mm.

A guide wire as shown in the figures, may conveniently be used to locate the catheter as may a guide catheter. The use of guide catheters and guide wires is well known in cardiovascular PTCA and other balloon applications. The guide catheter may be made from braided Pebex, PU or nylon. A guide catheter is particularly useful for transcardio crossing.

Preferably the degree of transfection is enhanced by the use of ultrasound. The source of ultrasound may be external to the animal being treated but preferably application of ultrasound is localised particularly by placing the source within the liver and preferably by incorporation into the catheter. Thus in some preferred embodiments the catheter is provided with an ultrasonic oscillator arranged to generate ultrasonic vibrations in the region of nucleic acid delivery. The catheter may for example be provided with a piezo-electric transducer or an array thereof. The ultrasonic oscillator is preferably arranged to generate a directional oscillation so as to allow it to be directed at the targeted liver cells, thus minimising the power required.

The above apparatus is suitable for all types of gene therapy and thus the nucleic acid with which it is desired to transfect the liver cells may be in the form of or may comprise any of the vectors suitable for delivery of nucleic acid to a cell in vivo. Suitable vectors may simply be naked nucleic acid or liposomes which encapsulate nucleic acid. Naked nucleic acid, e.g. in the form of a plasmid, is particularly suitable for transfection of cells and is preferred for use according to the present invention. Plasmids based on the test plasmid used by Liu et al. supra are suitable and as shown by Liu et al. liver specific promoters are not required but may be used to increase specificity of gene expression.

More complicated but equally suitable vectors for delivery of nucleic acid to the liver and thus for transfection of the liver cells are viral vectors. Viruses are very well suited for use in gene therapy since foreign or heterologous genes or coding sequences may be inserted into the viral genome. After infection of the cell by the virus, the foreign nucleic acid is delivered to the nucleus of the cell. While viruses are able to actively infect cells, the present method of hydrodynamic nucleic acid delivery results in a significantly increased “infection” rate and thus in effect an increase in the transfection rate and in the efficacy of the gene therapy. There are at least five classes of clinically available viral vectors, derived from (onco)retrovirus, lentivirus, adenovirus, adeno-associated virus and herpes virus. Those viral vectors whose genomes are integrated into the host cell DNA (oncoretroviruses and lentiviruses) may be preferred where stable genetic alteration in dividing cells is required. The other viruses mentioned persist in the cell nucleus as extrachromosomal episomes but are capable of mediating persistent transgene expression in non-proliferating cells. The most appropriate vector will depend on the particular gene therapy being attempted.

For convenience, the term “gene” is used herein to describe regions of nucleic acid not only that are transcribed into mRNA and translated into polypeptides (structural genes), but also those that are transcribed into RNA (e.g. rRNA, tRNA) and those that function as regulators of the expression of the former two types. Preferably the nucleic acid delivered to the liver will encode a structural gene relevant (directly or indirectly) to treatment of a given medical condition but it may be appropriate to introduce regulatory regions which, in combination with the genes already present in the cell, can provide a therapeutic benefit.

The nucleic acid molecule of the vector is typically DNA but may, for example where the vector is an RNA virus, be RNA. Antisense molecules and iRNA may be suitable for certain therapeutic regimen. Non-viral vectors may contain cDNA and the nucleic acid may be linear or circular, e.g. as with plasmid DNA. DNA may be single or double stranded.

Where the nucleic acid encodes a protein which it is desired to express in transfected cells, the nucleic acid molecule will typically also comprise an operably linked promoter and possibly other regulatory sequences. For certain vectors, in particular viral vectors, the nucleic acid will also encode structural and other proteins involved with the generation of further vectors which can go on to transfect other cells, e.g. the gag, pol and env genes of an adenovirus. The design and construction of expression vectors being familiar to be skilled man and well described in the literature.

A carrier or preparation compound may be injected prior to treatment to flush out the blood or help open the capillaries, suitable compounds being known in the art.

As in the methods described by Liu et al. supra, the present methods and the apparatus for use in such methods can be considered hydrodynamic methods of nucleic acid delivery. In other words a comparatively large volume of a liquid formulation containing nucleic acid (e.g. a DNA solution) is introduced rapidly into the vein. Thus, for humans and other animals of a similar size, between 100 ml and 1300 ml of liquid formulation may be introduced in a single, continuous or substantially continuous, administration. Volumes will depend on the age, sex and strength of the subject, for example a healthy young male may receive 800-1300 ml while an elderly woman may receive 200-600 ml. As shown in the Examples smaller volumes can be used. Volumes used for human subjects will typically be 50 ml or greater, preferably 75 ml or greater, more preferably 100 ml or greater, e.g. 150-350 ml.

The liquid formulation may comprise, in addition to the plasmid or other vector, any physiologically acceptable carrier, saline being particularly preferred. The concentration of the nucleic acid delivered will vary depending on the therapy proposed and may readily be optimised by the skilled man. Suitable dosages include between 5 and 50 mg, e.g. 10-30 mg of plasmid per 500 ml of saline; a typical dose suitable for most patients would be 20 mg of plasmid in 500 ml of saline. As shown in the Examples, smaller doses and volumes are also appropriate, preferably 5 mg or more, e.g. 5-20 mg, provided in the volumes discussed above.

The speed of injection will depend on the pressure to be generated. Typically, using a syringe based system, 500 ml of saline containing plasmid would be administered over ½-8, e.g. 1-3 mins. Clearly larger volumes would generally require more time but more important than delivery time is the pressure at which the nucleic acid is delivered. In a closed fluid system as described herein, the pressure as monitored in the reservoir will correspond to the pressure at the point of delivery in the liver. As shown in the Examples, particularly where smaller volumes are used, injection time may be less, e.g. 10-60 seconds, preferably 15-30 seconds, e.g. around 20 seconds.

After rapid delivery of the nucleic acid, the hepatic vein is typically maintained in its occluded state for between 2 and 20, preferably 5-15, e.g. around 10 minutes. Reduction in occlusion is preferably achieved gradually, e.g. by slow deflation of the balloon.

According to a further aspect the present invention provides the use of a nucleic acid molecule in the manufacture of a medicament for introduction into a region of the body of a subject, under pressure, and via a substantially occluded efferent vessel of said body region, to treat said subject by gene therapy.

More particularly the present invention provides the use of a nucleic acid molecule in the manufacture of a medicament for introduction into the liver of a subject, under pressure, and via a substantially occluded hepatic vein. Suitable medicaments are described above and will typically comprise saline. As discussed herein, the nucleic acid may be naked, e.g. a plasmid or contained within a liposomal, viral or other vector. The nucleic acid and thus the medicament containing it are introduced for the purpose of performing gene therapy on the subject, e.g. for cells (e.g. liver cells) within the subject. There are many specific therapies that may be performed in this way, including treatment of cancer (generally, not limited to the liver), liver cirrhosis and other liver diseases as well as conditions which are not manifested within the liver but may benefit from the generation in the liver of proteins encoded by the nucleic acid with which the liver cells are transfected. Diseases affecting other organs of the body and other regions of the body may also be treated in accordance with the invention.

Certain preferred embodiments of the invention will now be described, by way of example only, with reference to the accompanying drawings in which:

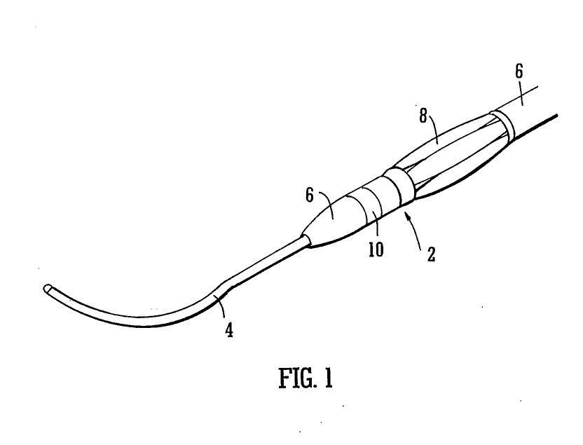

FIG. 1 is a perspective view of a catheter in accordance with the invention and associated guide wire;

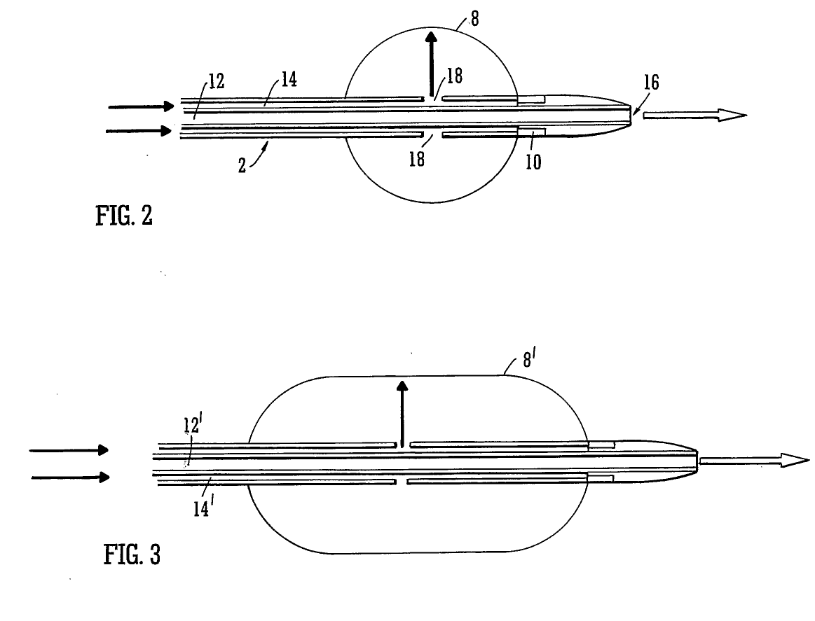

FIG. 2 is a sectional view through the catheter of FIG. 1;

FIG. 3 is a view similar to FIG. 2 of a slightly different embodiment;

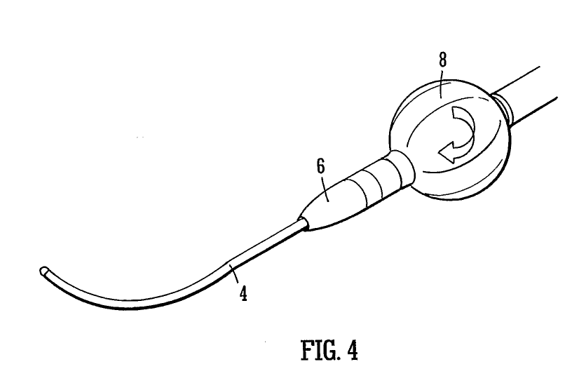

FIG. 4 is a view similar to FIG. 2 showing the balloon inflated;

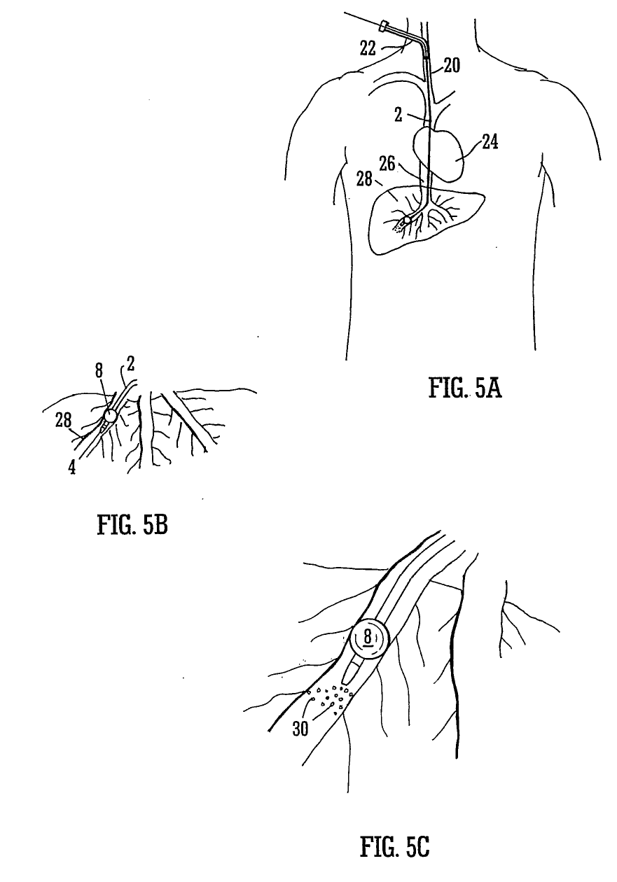

FIGS. 5a to 5c are schematic sectional views at varying levels of magnification showing the catheter being used;

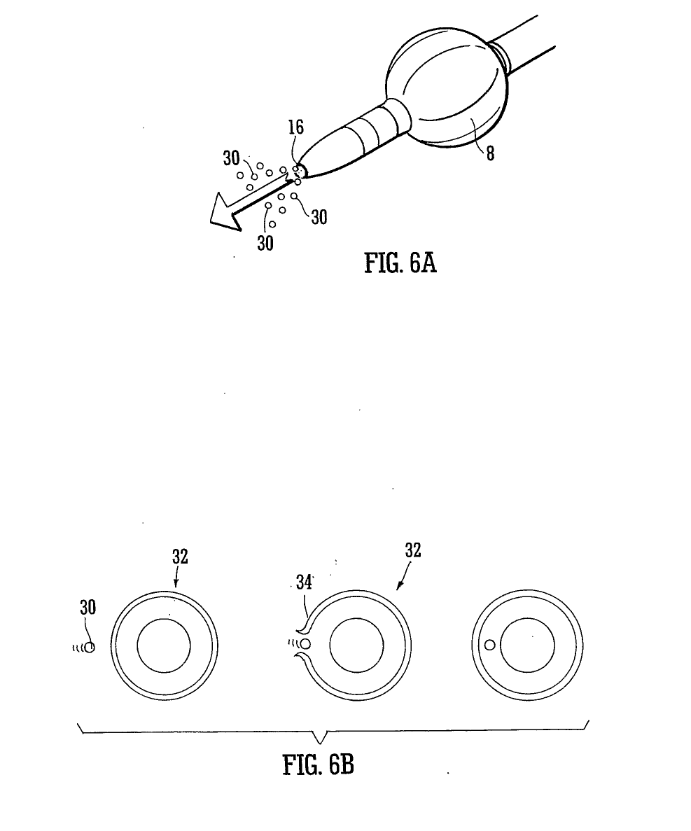

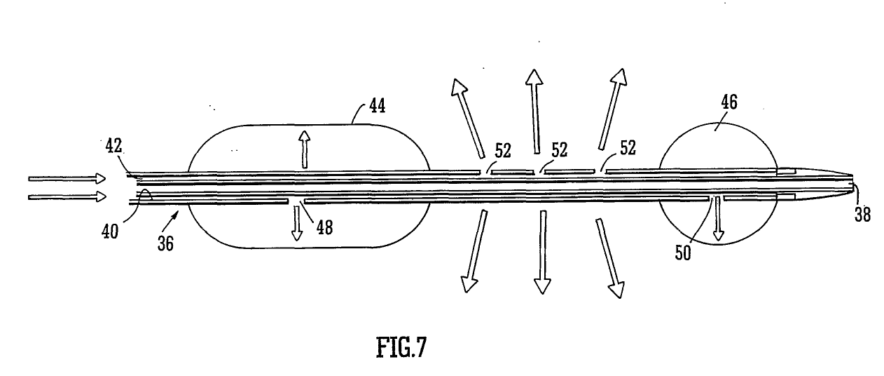

FIG. 6a is a view similar to FIG. 4 showing the pressurised introduction of nucleic acid (conveniently represented throughout as circularised);

FIG. 6b comprises a series of three schematic sectional views of transfection of a liver cell; and

FIG. 7 is a sectional view through a catheter in accordance with another embodiment of the invention.

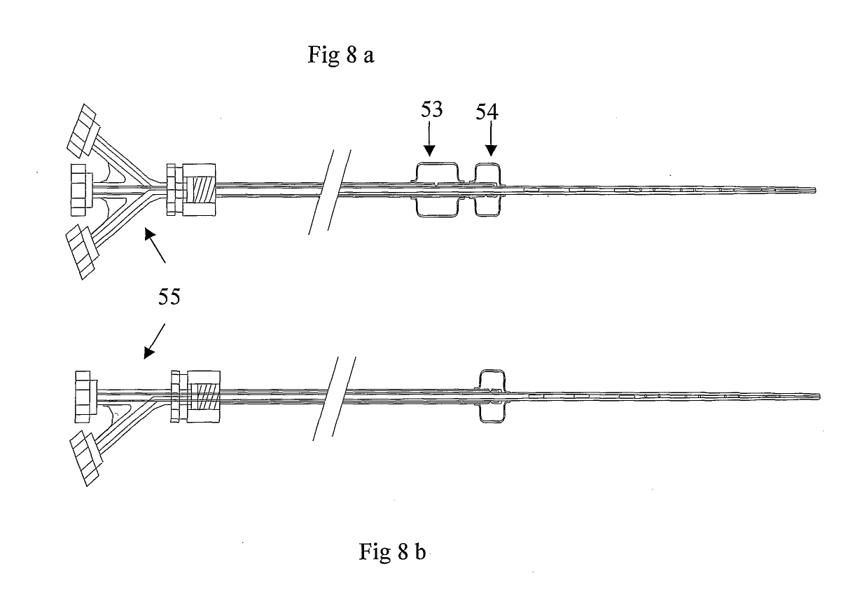

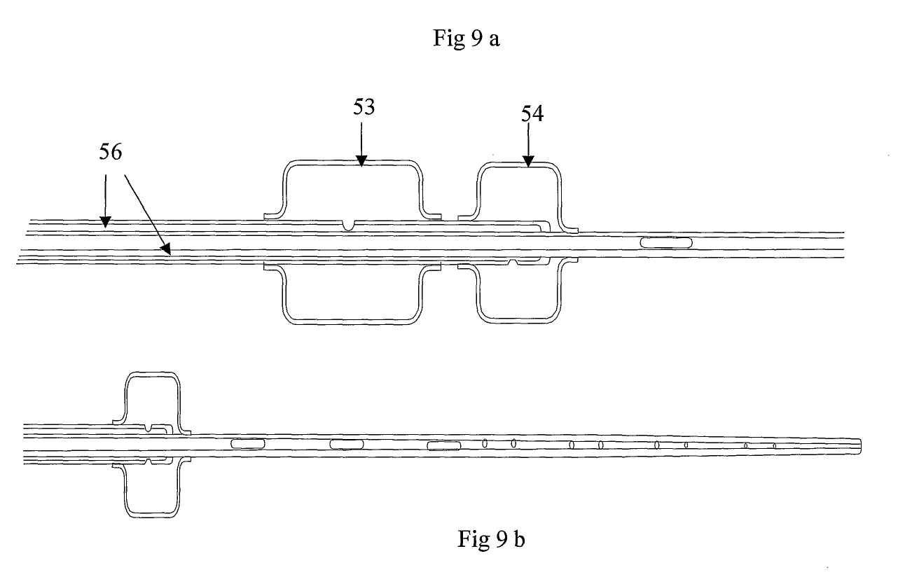

FIGS. 8a and 8b are sectional views through further catheters in accordance with further embodiments of the invention;

FIGS. 9a and 9b are close up sectional views of parts of FIGS. 8a and 8b;

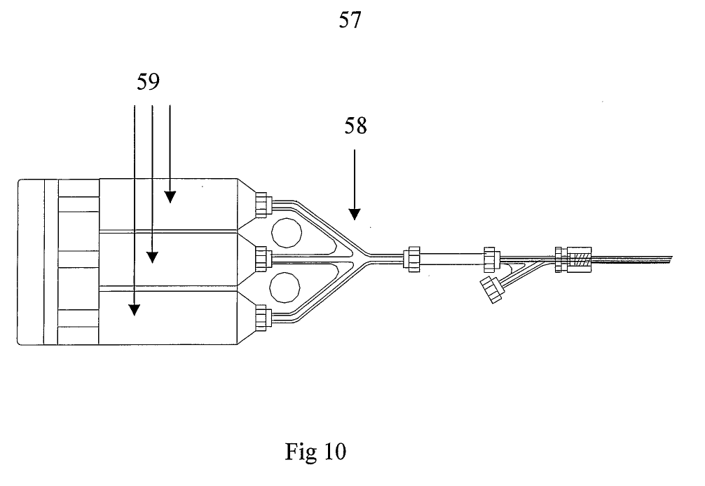

FIG. 10 is a sectional view of the injection system in accordance with the invention;

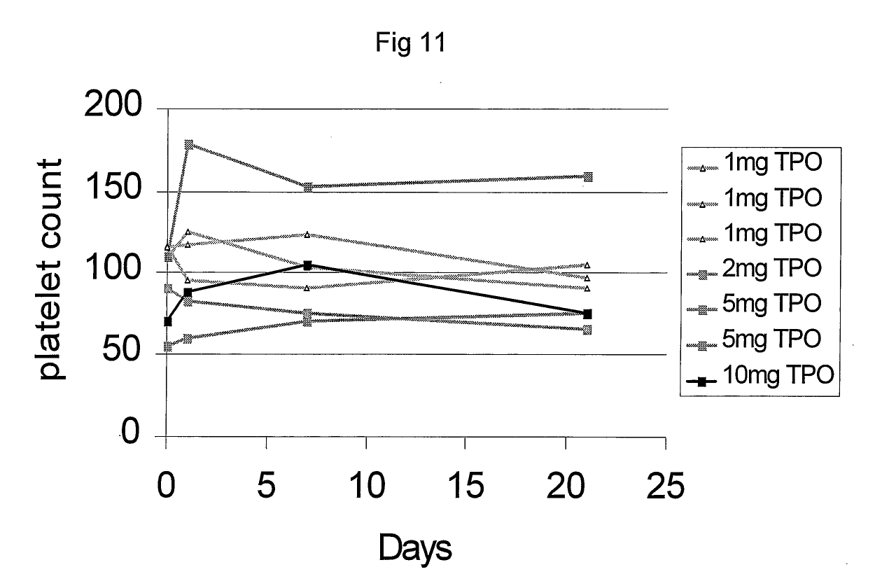

FIG. 11 is a graph showing the serum platelet count in 7 patients following the procedure performed in accordance with the invention and described in Example 4;

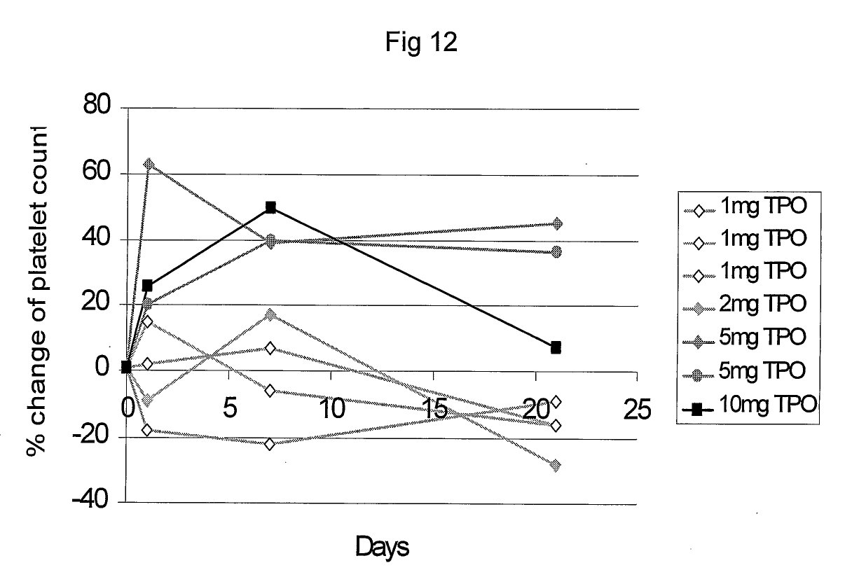

FIG. 12 is a graph based on the same data as FIG. 11 but showing the percentage change in platelet count compared to the base line.

Turning firstly to FIG. 1 there may be seen a catheter 2 in accordance with an embodiment of the invention having a corresponding guide wire 4 passing axially therethrough. The catheter 2 generally comprises an outer housing 6 which is divided longitudinally by an inflatable balloon 8. In the uninflated state shown in FIG. 1, the catheter and balloon is able to pass easily through the inferior vena cava via the heart and ascending vena cava.

A marker band 10 is provided around the foremost body section 6 in order to aid location in the body. The material of the marker band 10 will therefore depend upon the imaging system used.

FIG. 2 shows the catheter 2 in greater detail, with the guide wire omitted for clarity. It will be seen from this that the catheter 2 comprises two coaxial lumens 12, 14. The central lumen 12 opens out at the tip 16 of the catheter and in use receives the guide wire. The outer lumen 14 communicates with the interior of the balloon 8 by means of a circumferentially spaced series of apertures 18. The balloon 8 may therefore be inflated and deflated by introducing and withdrawing saline from the outer lumen 14. The skin of the balloon 8 is elastic and can be inflated up to a diameter of up to around 18 mm for an adult human, around 8 mm for a child depending upon the volume of saline inserted. This is larger than the diameter of the hepatic vein where the catheter will be used. FIG. 4 shows a perspective view of the balloon 8 in its inflated state.

FIG. 3 is a view similar to FIG. 2 showing a slightly different embodiment. This embodiment differs from that of FIG. 2 only in that the balloon 8′ is longitudinally extended as compared to the balloon 8 in FIG. 2. This may be advantageous in some circumstances as it will clearly have a greater area of contact with the vein wall and thus withstand a greater pressure without slipping for a given degree of inflation.

Use of the catheter described above in a method in accordance with the invention will now be described with additional reference to FIGS. 5a-5c and 6a-6b.

Referring initially to FIGS. 1, 2 and 5a, the guide wire 4 is inserted into the inferior vena cava 20 by means of an introducer 22 and then through the heart 24 into the ascending vena cava 26 and, into the right hepatic vein 28. The catheter 2 is then slid over the guide wire until the tip 16 thereof is located in the desired position in the hepatic vein 28. This may be achieved for example by monitoring the progress of the marker band 10 towards the tip of the catheter using an ultrasound or other suitable imaging system.

Once the tip 16 of the catheter is in position, saline is pumped into the outer lumen 14 in order to inflate the balloon 8 until it presses against the walls of the hepatic vein 28 which may be seen in FIG. 5b. This fixes the location of catheter 2 in the vein and occludes the flow of blood to the heart 24. The guide wire 4 may then be fully or partly withdrawn. Thereafter a liquid formulation containing nucleic acid material for the required gene therapy is injected through the central lumen 12 of the catheter at a controlled pressure. In this embodiment the required pressure is achieved using a pre-programmed syringe driver although many suitable ways of achieving this may be envisaged.

The ejection of the schematically-depicted nucleic acid 30 is shown in FIGS. 5c and 6a. The occlusion of the hepatic vein 28 by the catheter balloon 8 retains the nucleic acid 30 at pressure within the liver rather than allowing it to travel up the ascending vena cava 26 to the heart 24. In a particular example the nucleic acid is introduced at a pressure of approximately 50 mmHg which pressure is withstood by the action of the balloon 8 on the walls of the vein 28.

The effect of this pressurised nucleic acid on the liver cells 32 in this area of the liver is to force the nucleic acid 30 through the walls 34 of the liver cells as is shown schematically in FIG. 6b, which then means that the nucleic acid is taken up by the cell 32 thereby allowing the nucleic acid to exert its influence on the cell's protein production.

In one example, the therapy is continued in this manner for up to 10 minutes, preferably 1 to 5 minutes and a volume of between 100 ml and a litre is administered depending upon the relative strength of the patient.

Once administration has finished and typically after a further period of 5-20, e.g. 10 mins, the guide wire 4 is replaced down the central lumen 12, the balloon 8 is deflated by withdrawing saline therefrom. This allows blood and some of the introduced liquid to flow to the heart 24. The catheter 2 is then removed by sliding it over the guide wire 4 and finally the guide wire 4 is removed.

Thus in accordance with the described apparatus and methods, an improved method of gene therapy exhibiting significantly higher transfection efficiencies in hepatic liver cells is disclosed.

A further embodiment of the invention is shown in FIG. 7. In this embodiment, the catheter 36 comprises three lumens. In addition to a central guide wire lumen 38, there are upper and lower side lumens 40, 42. The lower side lumen 40 communicates with a pair of axially spaced balloons 44, 46 by means of corresponding side apertures 48, 50. The upper side lumen 42 opens out radially in a series of side apertures 52 located axially between the two balloons 44, 46.

Use of the catheter 36 shown in FIG. 7 is similar to the previous embodiment except that since the nucleic acid is not administered through the guide wire lumen 38, there is no need to withdraw the guide wire (not shown for clarity) during the procedure. Furthermore, the provision of two balloons 44, 46 allows a section of the hepatic vein to be fluidically isolated both upstream and downstream which means that the gene delivery is not affected by blood flow at all and may mean that a higher administration pressure can safely be used as compared to the previous embodiment.

Further embodiments of the invention are shown in FIGS. 8a and b and 9a and b. In FIGS. 9a and 9c the first balloon 53 can act as a pressure dam while the second balloon 54 effects the occlusion. The lumen are capped by standard hemostasis valve Y junctions 55. The Y junction allows the insertion of a guidewire and inflation ports. The valve is a silicone seal or “O” ring which closes down on to a taper when the end cap is twisted, this closes the lumen. The valve stops blood and fluid loss along the central lumen used for the guide wire and delivery of the nucleic acid. The dual inflation lumen 56 shown clearly in FIG. 9a allow different inflation pressures.

FIG. 10 shows an embodiment of an injection system 57 in accordance with the invention which is able to deliver 300 ml of liquid in 12 seconds. A manifold 58 is provided to which are attached three syringes 59.

It will be appreciated by those skilled in the art that only certain preferred embodiments of the invention have been described and that there are many variations and modifications possible within the scope of the invention. For example, a centrally guided catheter is not essential and for example a monorail catheter could be used instead. It is also envisaged that the cells undergoing the described therapy may be subjected to ultrasound or other suitable form of radiation in order to enhance the transfection thereof by the nucleic acid. An ultrasonic vibrator e.g. a piezo-electric oscillator could be provided on the catheter for this purpose.

The invention is further described in the following Examples:

### EXAMPLE 1

The following protocol was performed on 2 pigs of around 40 kg.

The pigs were put under general anaesthetic. A catheter was introduced in the neck vein (external jugular). The catheter had 2 channels; one central channel that can carry an introducer (e.g. a guide wire) and another that can be used to inflate a balloon.

The catheter was pushed down from the neck veins under image intensifier to the superior vena cava, right heart, supra-hepatic vena cava until it reached one of the 3 hepatic veins. For the purpose of this experiment the left hepatic vein is the most suitable.

It was introduced until the catheter did not advance any further.

The balloon was then inflated in order to close completely the lumen of the hepatic vein.

Then the introducer was removed and the nucleic acid injected fast, within a minute or two, under pressure. A volume of 500-1000 ml was injected.

The balloon was kept inflated for about 10 minutes, then deflated slowly and the catheter removed.

The anaesthetic was then discontinued and the animal was recovered. Serial blood tests were performed for 3 weeks to check on any toxicity, liver damage as well as gene expression.

These experiments have shown that this technique was safe. The liver function test remained normal and the animal remained in good health. Significant gene expression was observed.

### EXAMPLE 2

In this example the plasmid pDERM II expressing rat TPO (thrombopoietin) under the control of a liver specific promoter was injected into the hepatic vein of rats after inferior vena cava (IVC) occlusion and intravenously into the tail vein of rats (controls). 400 g rats were injected with 100 μg of plasmid. The IVC was clamped just above or in the junction with hepatic veins.

TPO is normally produced in the liver and acts on the bone marrow where it stimulates production of platelets by megakaryocytes. The count of platelets (PLT) and white blood cells (WBC) in 1 ml of blood in the systemic circulation were measured in 7 rats and the mean values for each group calculated. The results are shown in Table 1 below, all values are in thousands.

TABLE 1
DAY 7

| | |
|---|---|
| Controls | pDERM TPO |

| PLT | WBC | PLT | WBC |
|---|---|---|---|
| 1239 | 5.5 | 1416 | 7.8 |
| 895 | 6.7 | 1388 | 7.9 |
| 926 | 6.8 | 1449 | 7.4 |
| | | 1411 | 7.4 |
| 987 | 6 | 1416 | 7.6 |

These results show that levels of TPO, i.e. plasmid transfection efficiency, are greater where hydrodynamic injection into the hepatic vein is used.

### EXAMPLE 3

### Background

Patients with Hepatitis C, liver cirrhosis suffer from thrombocytopenia (i.e. low platelet count]. Thrombopoietin (TPO) is secreted from the liver and circulates to the bone marrow and leads to the maturation of megakaryocytes and results in platelet release. Patients with liver cirrhosis have low TPO production and it is proposed to use gene therapy to augment the TPO production in order to bring back the platelet count to normal levels.

Prior to initiating a clinical study we studied the feasibility of this approach in pigs using the hydrodynamic technique of the present invention

### Animals & Methods

Four pigs (median weight 50 kgs) were studied. Prior to gene therapy injection they underwent haematological (full blood count), biochemical (liver function tests, urea and electrolytes as well as serum alpha feto protein measurements) and radiological investigations (ultrasound scan).

Under general anaesthetic and endo-tracheal ventilation a catheter was introduced in the hepatic vein via the internal jugular vein. A contrast material was injected in the catheter after inflation of the balloon in order to verify that the catheter balloon was completely obstructing the hepatic vein and did not allow reflux towards the vena cava.

Three pigs were injected with a plasmid encoding human TPO under the control of a liver specific promoter dissolved in normal saline. This was injected over 20 seconds into the obstructed liver segment. TPO plasmid was injected in a dose of 10 mgs dissolved in 200 mls of normal saline. The fourth pig was injected with a plasmid encoding lac Z which gives blue colouration with beta gal staining.

In each case a single injection was performed. Post-injection blood tests were made in order to assess haematological, biochemical and liver parameters.

### Results

TABLE 2

| Pig A | Day 0 | Day 2 | Day 3 | Day 3 | Day 7 | Day 13 |
|---|---|---|---|---|---|---|
| Platelets 10.9/L | 280 | | | 355 | 330 | 340 |
| White blood cells 10.9/L | 8 | | | 22 | 18 | 18 |
| Bilirubin total umol/L | 16.8 | 10.1 | 10.7 | 6 | | |
| Bilirubin umol/L | 4.2 | 7 | 3.7 | 2.6 | | |
| ASAT (TGO) umol/L | 47 | 44 | 70 | 55 | | |
| ALAT (TGP) umol/L | 38 | 45 | 46 | 46 | | |
| γ GT UI/L | 20 | 24 | 34 | 26 | | |
| Ph. Alc. UI/L | 128 | 113 | 91 | 78 | | |
| Total protein g/L | 58 | 64 | 64 | 64 | | |
| Albumin g/L | 21 | 23 | 23 | 23 | | |
| Amylases UI/L | 1097 | 1148 | 1022 | 1082 | | |
| Sodium mmol/L | 141 | 141 | 141 | 139 | | |
| Potassium mmol/L | 3 | 3.8 | 3.7 | 3.6 | | |
| Chlorine mmol/L | 98 | 101 | 102 | 101 | | |
| Glucose mmol/L-(gr/L) | 5 (0.9) | 5.8 (1.04) | 4.9 (0.88) | 4.9 (0.88) | | |
| Urea mmol/L | 3.2 | 4.5 | 2.7 | 2.6 | | |
| Creatine umol/L-(mg/L) | 67 (7.6) | 89 (10.1) | 95 (10.7) | 91 (10;3) | | |

TABLE 3

| Pig B | Day 0 | Day 0 | Day 3 | Day 3 | Day 7 | Day 13 |
|---|---|---|---|---|---|---|
| Platelets 10.9/L | 560 | 340 | 424 | 402 | 622 | 413 |
| White blood cells 10.9/L | 9.9 | 10.2 | 26.5 | 25.8 | 15.1 | 19.6 |
| Red blood cells 10.12/L | 4.93 | 5.11 | 5.03 | 5.06 | 5.63 | 5.38 |
| Haematocrit % | 26 | 27 | 27 | 26 | 29 | 28 |
| Haemoglobulin g/dl | 8.8 | 9.1 | 9 | 9.1 | 10.2 | 9.6 |
| Prothrombin % | 98 | 98 | 100 | ND | ND | ND |
| Fibrinogen g/L | 2.05 | 2.17 | 2.63 | ND | ND | ND |
| Bilirubin total umol/L | 8.7 | 8.6 | ND | 5.6 | ND | ND |
| Bilirubin (conjugate) umol/L | 2.8 | 2.8 | ND | 2.5 | ND | ND |
| ASAT (TGO) umol/L | 29 | 29 | ND | 42 | ND | ND |
| ALAT (TGP) umol/L | 31 | 31 | ND | 39 | ND | ND |
| γ GT UI/L | 19 | 20 | ND | 80 | ND | ND |
| Ph. Alc. UI/L | 157 | 159 | ND | 106 | ND | ND |
| Total protein g/L | 58 | 58 | ND | 71 | ND | ND |
| Albumin g/L | 19 | 19 | ND | 21 | ND | ND |
| Amylases UI/L | 910 | 914 | ND | 998 | ND | ND |

TABLE 4

| Pig C | Day 0 | Day 0 | Day 3 | Day 3 | Day 7 | Day 13 |
|---|---|---|---|---|---|---|
| Platelets 10.9/L | 520 | 528 | 474 | 431 | 679 | 617 |
| White blood cells 10.9/L | 14.4 | 14.4 | 32.3 | 27.6 | 27.8 | 25 |
| Red blood cells 10.12/L | 5.5 | 5.55 | 5.56 | 5.59 | 5.92 | 5.94 |
| Haematocrit % | 26 | 27 | 26 | 27 | 29 | 28 |
| Haemoglobulin g/dl | 8.8 | 8.8 | 8.9 | 8.8 | 9.6 | 9.6 |
| Prothrombin % | 100 | 100 | 100 | ND | ND | ND |
| Fibrinogen g/L | 2.41 | 2.44 | 3.74 | ND | ND | ND |
| Bilirubin total umol/L | 9.8 | 9.5 | ND | 5.8 | ND | ND |
| Bilirubin conjugate umol/L | 2.9 | 3 | ND | 2.4 | ND | ND |
| ASAT (TGO) umol/L | 26 | 25 | ND | 35 | ND | ND |
| ALAT (TGP) umol/L | 29 | 32 | ND | 36 | ND | ND |
| γ GT UI/L | 22 | 22 | ND | 30 | ND | ND |
| Ph. Alc. UI/L | 180 | 182 | ND | 106 | ND | ND |
| Total protein g/L | 63 | 62 | ND | 69 | ND | ND |
| Albumin g/L | 19 | 18 | ND | 19 | ND | ND |
| Amylases UI/L | 1692 | 1672 | ND | 1541 | ND | ND |

As shown in the tables there were no complications associated with this procedure. There were no significant changes in the liver function tests and there was an increase in both platelet count and white blood cells.

Blue colouration in the liver following injection of plasmid lac Z was further evidence of successful transfection.

Plasmid TPO injected according to the method of the invention with doses of 10 mgs and above with a voume in excess of 50 mls can lead to increased serum platelet count and white blood cells. It is proposed that this approach could be used in all forms of liver gene therapy.

### EXAMPLE 4

### Background

In previous pre-clinical models we have shown that it was difficult to increase significantly the TPO levels without the hydrodynamic technique of the present invention. Example 3 shows that our hydrodynamic technique can increase significantly TPO production in a large animal such as pigs (weight over 50 kg).

Therefore a clinical study was initiated in patients with thrombocytopenia to find out whether gene therapy with plasmid TPO injected with the hydrodynamic technique of the present invention can increase the platelet count.

### Patients & Methods

Seven patients (2 males and 5 females), median age 52 yrs were studied. Prior to gene therapy injection they underwent haematological (full blood count), biochemical (liver function tests, urea and electrolytes as well as serum alpha feto protein) and radiological investigations (ultrasound and CT scans).

Following signature of informed consent a catheter was introduced in the hepatic vein via the femoral vein under local anaesthetic. A contrast material was injected in the catheter after inflation of the balloon in order to verify that the catheter balloon is completely obstructing the hepatic vein and does not allow reflux towards the vena cava.

Plasmid TPO dissolved in normal saline was injected for 20 seconds into the obstructed liver segment. The injection was performed by hand, fast and forcefully. TPO plasmid was injected at a dose of 1 mg in patients 1, 2 & 3, in 50 ml, 75 ml and 100 ml respectively. Patient 4 was injected with 2 mg in 150 ml. Patients 5 and 6 were injected with 5 mgs in 150 ml and 200 ml respectively. The seventh patient was injected with 10 mgs in 200 ml and the eighth patient with 10 mg in 250 ml. The balloon was deflated 5 minutes following the injection and the catheter was removed afterwards. Patients were discharged home 2 hours following this procedure. In each case a single injection was performed.

Post-injection blood tests were made in order to assess haematological, biochemical and liver parameters.

### Results

There were no complications associated with this procedure. There was no fever or rigors. There was minimal pain in the groin just during the catheter insertion. There were no changes in the liver function tests. FIG. 11 shows the serum platelet count in the first seven patients. FIG. 12 shows the percentage change in platelet count compared to the base line. These results show that the platelet count did not change in the first 4 patients that received 1 or 2 mgs plasmid TPO. On the other hand it is quite clear the patients 5, 6 and 7 which received 5 and 10 mgs did have a 40 to 60% increase in the platelet count which lasted over 3 weeks.

### Conclusion

Thus plasmid TPO injected in accordance with the present invention with doses of 5 mg and above and at a volume in excess of 50 ml can lead to increased serum platelet count. This approach potentially could be used in all forms of liver gene therapy.

1. A method for introducing nucleic acid into cells of a region of the human or animal body, which method comprises substantially occluding an efferent vessel from said body region and introducing said nucleic into that body region under pressure via said efferent vessel.

2. Apparatus for introducing nucleic acid into cells of a region of the body comprising: a reservoir for holding a liquid formulation which comprises said nucleic acid; a catheter tube in fluid communication with said reservoir for conveying said liquid formulation to said body region via an efferent vessel of said body region; pressure development means for pressurising the liquid conveyed by the catheter; and occlusion means for substantially occluding said efferent vessel.

3. A method of claim 1 wherein said region of the body is an organ of the body.

4. A method of claim 3 wherein the organ is selected from the list comprising kidney, heart, spleen, pancreas, lung, adrenal glands, stomach, prostate gland and ovary.

5. A method of claim 4 wherein the organ is the liver.

6. A method of claim 1 wherein the nucleic acid is introduced at a pressure of, or the pressure development means are adapted to generate a pressure of, 10-80 mmHg.

7. A method of claim 1 wherein the nucleic acid is in the form of a plasmid.

8. A method of claim 1 wherein occlusion is achieved by or the occluding means comprises one or more balloons.

9. A method of claim 1 wherein the nucleic acid is introduced into said region of the body in less than 60 seconds.

10. A method of claim 1 wherein the liquid formulation comprising said nucleic acid has a total volume of 50-1300 ml.

11. A method of claim 10 wherein the liquid formulation comprising said nucleic acid has a total volume of 75-350 ml.

12. Apparatus of claim 2 wherein said reservoir comprises one or more syringe tubes.

13. Apparatus of claim 2 wherein said pressure development means comprises one or more syringes.

14. Apparatus of claim 2 wherein said catheter comprises one or more radial injection ports.

15. Apparatus of claim 2 wherein said catheter comprises 2 lumen.

16. Apparatus of claim 15 wherein one lumen is adapted to receive a guide wire.

17. Apparatus of claim 15 wherein one lumen is adapted to allow inflation of the occlusion means.

18. Apparatus in of claim 16 wherein the liquid formation passes down the guide wire lumen.

19. Apparatus of claim 15 wherein the catheter comprises two lumen which are adapted to allow inflation of the occlusion means.

20. (canceled)
